(19) United States
(12) Patent Application Publication
(10) Pub. No.: US 2019/0021612 A1
(43) Pub. Date: Jan. 24, 2019

(54) WRIST SPHYGMOMANOMETER

(71) Applicant: HEALTH & LIFE CO., LTD., New Taipei City (TW)

(72) Inventors: Meng-Yi LIN, New Taipei City (TW); Wei-Te LIN, New Taipei City (TW); Chun-Ying LI, New Taipei City (TW)

(21) Appl. No.: 15/967,094

(22) Filed: Apr. 30, 2018

(30) Foreign Application Priority Data
Jul. 20, 2017 (TW) 106210656

(51) Int. Cl. A61B 5/021; A61B 5/0225; A61B 5/00

(52) U.S. Cl. CPC A61B 5/02141; A61B 5/0225; A61B 2560/0406; A61B 5/742; A61B 5/681

(57) ABSTRACT

The wrist sphygmomanometer includes a wristband, an inflatable bladder, a measuring unit and a pressure sensing unit. The wristband has an inner surface that is adapted to contact a human wrist. The inflatable bladder is mounted inside the wristband, and has a measurement point adapted to correspond in position to a point of radial pulse on the human wrist. The measuring unit is mounted on an outer surface of the wristband and is connected to the inflatable bladder. The pressure sensing unit includes a pressure sensor mounted at the measurement point of the inflatable bladder, and a wire electrically interconnecting the measuring unit and the pressure sensor.

### CROSS-REFERENCE TO RELATED APPLICATION

This application claims priority of Taiwanese Patent Application No. 106210656, filed on Jul. 20, 2017.

### FIELD

The disclosure relates to a sphygmomanometer, and more particularly to a wrist sphygmomanometer.

### BACKGROUND

Blood pressure is one of vital signs indicating health status of an individual's circulatory system. Proper monitoring of blood pressure allows the individual to take preemptive measures against cardiovascular diseases such as stroke, heart failure and hypertensive heart disease.

Portable forms of sphygmomanometers, notably wrist blood pressure monitors, have been commonly used to assist an individual to take measurement on a regular basis. Referring to FIG. 1, a conventional wrist sphygmomanometer 6 includes a wristband 61 and a measuring unit 62 mounted on the wristband 61. The wristband 61 is adapted to be worn around a human wrist 7 and includes a gas chamber 611 therein. The measuring unit 62 includes a pressure sensor (not shown) and a inflation unit (not shown) used to pressurize and depressurize the gas chamber 611. In order to ensure accuracy of blood pressure reading, the wristband 61 is tightly attached around the wrist 7, such that the measuring unit 62 is proximate to an inner side 71 of the wrist 7. After the inflation unit of the measuring unit 62 inflates the gas chamber 611 of the wristband 61, the pressure sensor conducts blood pressure measurement through interfered radial pulse of the wrist 7.

Despite its portable design, the wrist sphygmomanometer 6 is uncomfortable to be worn for a long duration. In a circumstance in which hourly measurements of blood pressure has to be taken on a daily basis, if the individual wishes to remain comfortable between the measurements, the individual would have to inconveniently put on, measure with, and take off the wrist sphygmomanometer 6 every time during each measurement, which might be equally undesirable as well.

### SUMMARY

Therefore, an object of the disclosure is to provide a wrist sphygmomanometer that can alleviate at least one of the drawbacks associated with the abovementioned prior art.

The wrist sphygmomanometer is adapted to be worn by a human wrist for blood pressure monitoring. The human wrist has an inner side at which a point of radial pulse is located, and an outer side which is opposite to the inner side. The wrist sphygmomanometer includes a wristband, an inflatable bladder, a measuring unit and a pressure sensing unit.

The wristband has an inner surface that is adapted to contact the human wrist, and an outer surface that is opposite to the inner surface. The outer surface has first and second surface sections adapted to be respectively proximate to the outer and inner sides of the human wrist. The inflatable bladder is mounted inside the wristband between the inner surface and the outer surface, and has a measurement point that is proximate to the second surface section of the outer surface and this is adapted to correspond in position to the point of radial pulse on the human wrist. The measuring unit is mounted on the first surface section of the outer surface of the wristband, and is connected to the inflatable bladder. The pressure sensing unit includes a pressure sensor that is mounted at the measurement point of the inflatable bladder, and a wire that electrically interconnects the measuring unit and the pressure sensor. The measuring unit is operable to measure, via the pressure sensing unit, a pressure inside the inflatable bladder at the measuring point.

### BRIEF DESCRIPTION OF THE DRAWINGS

Other features and advantages of the disclosure will become apparent in the following detailed description of the embodiments with reference to the accompanying drawings, of which:

FIG. 1 is a front view of a conventional wrist sphygmomanometer in use;

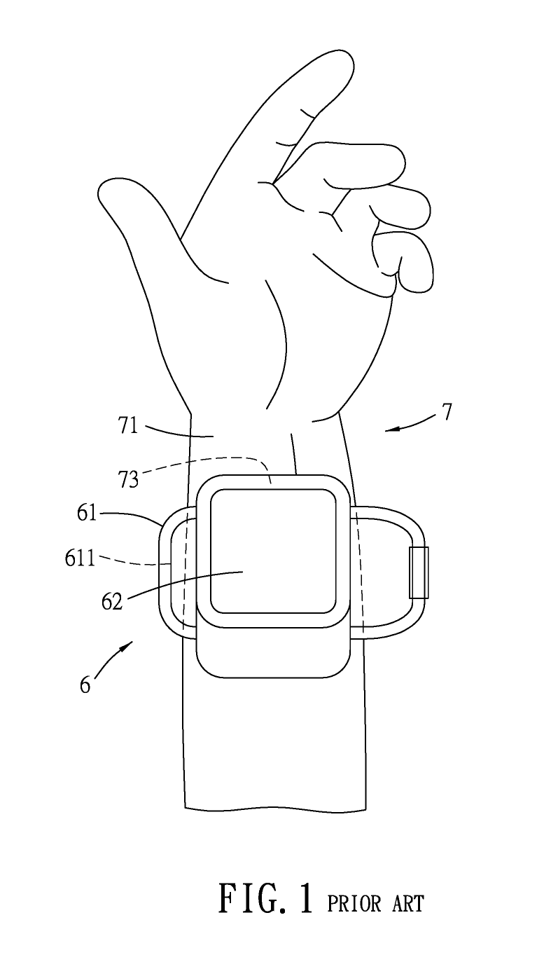

FIG. 2 is a side view of an embodiment of an wrist sphygmomanometer according to the present disclosure worn by a human wrist;

FIG. 3 is another side view of the embodiment; and

FIG. 4 is an exploded perspective view of the embodiment.

### DETAILED DESCRIPTION

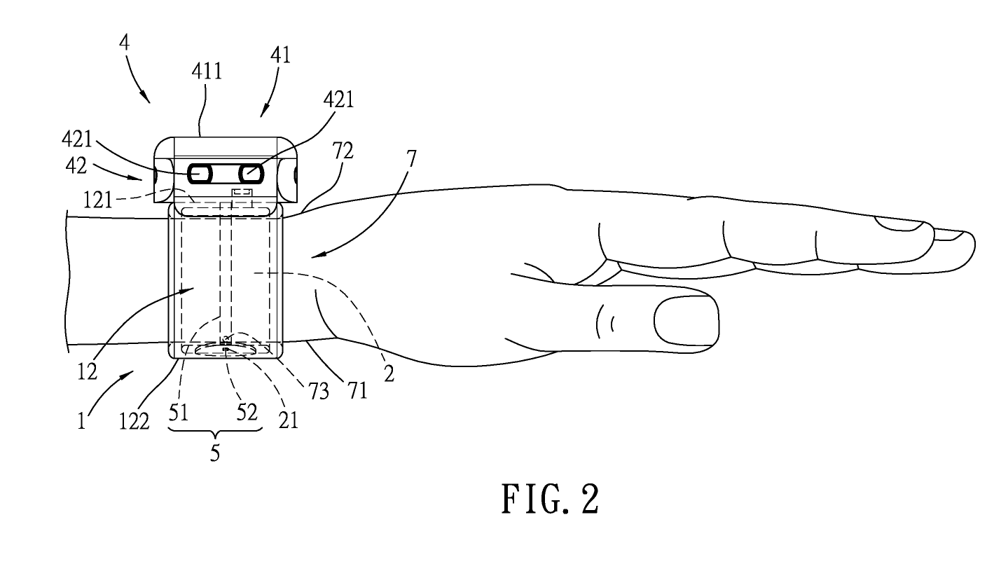

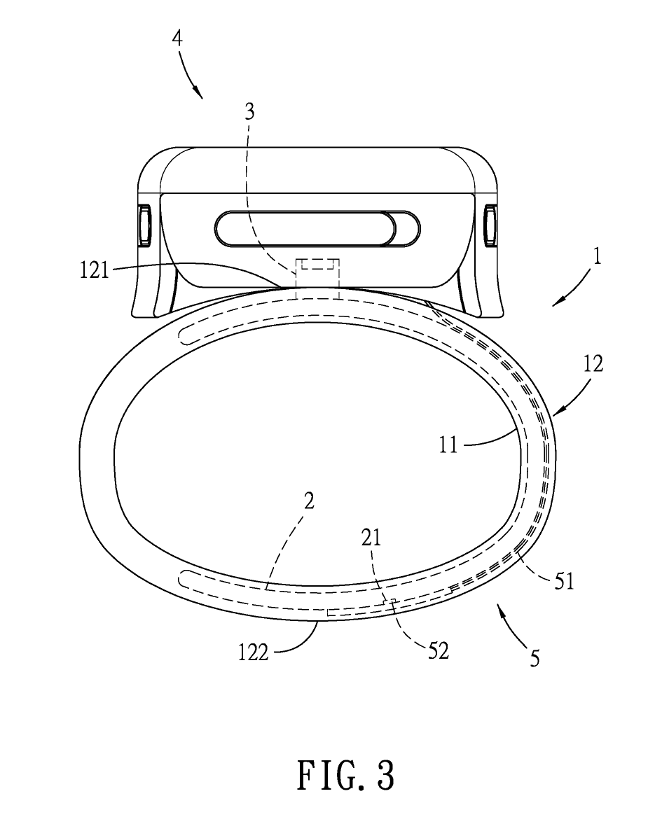

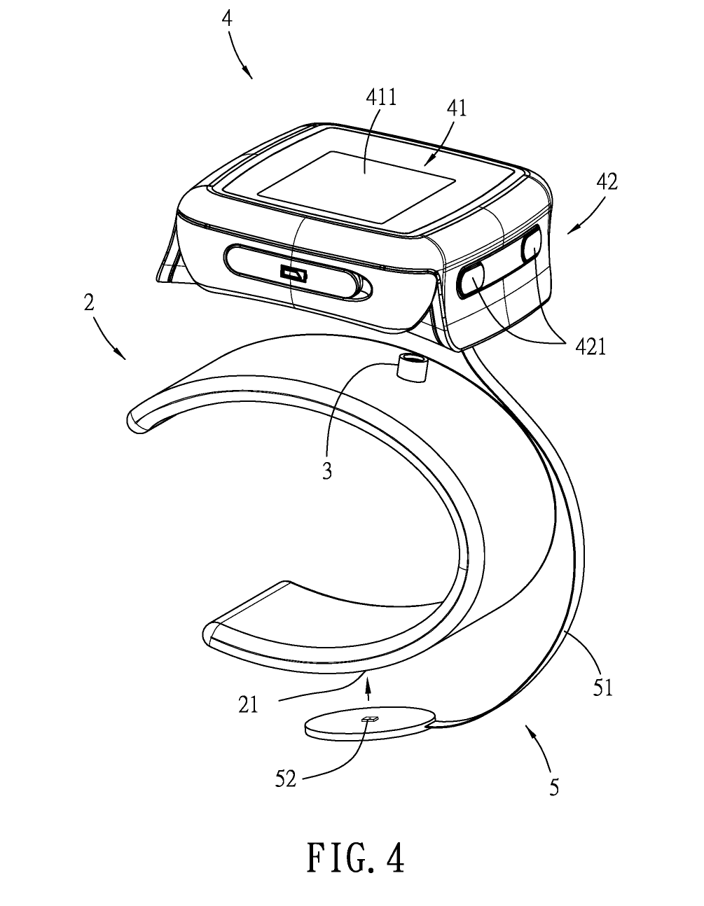

Referring to FIGS. 2 to 4, an embodiment of a wrist sphygmomanometer of this disclosure is adapted to be worn by a human wrist 7 for blood pressure monitoring. The human wrist 7 has an inner side 71 at which a point of radial pulse 73 is located, and an outer side 72 which is opposite to the inner side 71. The wrist sphygmomanometer includes a wristband 1, an inflatable bladder 2, a valve 3, a measuring unit 4, and a pressure sensing unit 5.

The wristband 1 is adapted to be worn by the human wrist 7, and has an inner surface 11 and an outer surface 12 that is opposite to the inner surface 11. The inner surface 11 is adapted to contact the human wrist 7, and the outer surface 12 has first and second surface sections 121, 122 adapted to be respectively proximate to the outer and inner sides 72, 71 of the human wrist 7.

The inflatable bladder 2 is mounted inside the wristband 1 between the inner surface 11 and the outer surface 12, and has a measurement point 21 that is proximate to the second surface section 122 of the outer surface 12 and that is adapted to correspond in position to the point of radial pulse 73 on the human wrist 7. The valve 3 extends outwardly from the inflatable bladder 2 through the first surface section 121 of the wristband 1 and is in fluid communication with the inflatable bladder 2.

The measuring unit 4 is mounted on the first surface section 121 of the outer surface 12 and is connected to the valve 3. The measuring unit 4 includes a display unit 41, an operation unit 42, an inflation unit (not shown) and a processing unit (not shown). The display unit 41 has a display screen 411 on top of the measuring unit 4 that is operable to display measured value such as blood pressure and heart rate. The operation unit 42 has a plurality of buttons 421 at a side of the measuring unit 4 that are operable of initiating basic tasks of the wrist sphygmomanometer such as taking measurement and turning power on and off. The inflation unit and the processing unit are inside the measuring unit 4, in which the inflation unit is operable to pump and release air into the inflatable bladder 2 through the valve 3. The processing unit receives signals from the operation unit 42 to control the display unit 41 and the inflation unit.

The pressure sensing unit 5 includes a pressure sensor 52 that is mounted at the measurement point 21 of the inflatable bladder 2, and a wire 51 that electrically interconnects the measuring unit 4 and the pressure sensor 52. In this embodiment, the pressure sensing unit 5 is disposed in the wristband 1 at one side of the inflatable bladder 2 which is proximate to the outer surface 12 to prevent outside interference. Placement of the pressure sensing unit 5 is not restricted to such and may be disposed outside of the wristband 1 in other embodiments as well. The wire 51 in this embodiment is made of flexible printed circuit board, but is not limited to such, that is operable to transmit signals between the measuring unit 4 and the pressure sensor 52. In actual operation, placement of the pressure sensor 52 may be freely adjusted to correspond in position to the point of radial pulse 73 on the human wrist 7 of different users. As the pressure sensing unit 5 extends to the measurement point 21 directly from the measuring unit 4, the measurement unit 4 is operable to measure, via the pressure sensing unit 5, a pressure inside the inflatable bladder 2 at the measurement point 21 as well as signal transmitted from the point of radial pulse 73 to the inflatable bladder 2, such that pressure difference or pressure loss due to folding of the inflatable bladder 2 around the human wrist 7 do not undermine actual reading of the blood pressure and heart rate.

A typical operation of the wrist sphygmomanometer is as follows, but is not limited to such:

After a wrist sphygmomanometer is placed around the human wrist 7, the pressure sensor 52 is adjusted to correspond in position to the point of radial pulse 73 of the human wrist 7. Then, after turning on the measuring unit 4, one of the buttons 421 on the operation unit 42 designated to measure blood pressure is pressed to initiate blood pressure measurement, in which the inflation unit of the measuring unit 4 inflates the inflatable bladder 2, allowing the pressure sensor 52 of the pressure sensing unit 5 to measure pressure disturbance within the inflatable bladder 2.

In summary, the wrist sphygmomanometer of this disclosure, through the design of the pressure sensing unit 5, is operable to measure accurate blood pressure of a user while being comfortable to be worn for a long duration without interfering with the user's daily routine. In addition, as the pressure sensing unit 5 extends to the measurement point 21 directly from the measuring unit 4, a pressure inside the inflatable bladder 2 at the measurement point 21 as well as signal transmitted from the point of radial pulse 73 to the inflatable bladder 2 may be directly measured to give more accurate reading of blood pressure and heart rate.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A wrist sphygmomanometer adapted to be worn by a human wrist for blood pressure monitoring, the human wrist having an inner side at which a point of radial pulse is located, and an outer side which is opposite to the inner side, said wrist sphygmomanometer comprising:
a wristband having
an inner surface that is adapted to contact the human wrist, and
an outer surface that is opposite to said inner surface, and that has first and second surface sections adapted to be respectively proximate to the outer and inner sides of the human wrist;
an inflatable bladder mounted inside said wristband between said inner surface and said outer surface, and having a measurement point that is proximate to said second surface section of said outer surface and that is adapted to correspond in position to the point of radial pulse on the human wrist;
a measuring unit mounted on said first surface section of said outer surface of said wristband, and connected to said inflatable bladder; and
a pressure sensing unit including a pressure sensor that is mounted at said measurement point of said inflatable bladder, and a wire that electrically interconnects said measuring unit and said pressure sensor;
wherein, said measuring unit is operable to measure, via said pressure sensing unit, a pressure inside said inflatable bladder at said measuring point.

2. The wrist sphygmomanometer as claimed in claim 1, wherein said pressure sensing unit is disposed in said wristband at one side said inflatable bladder which is proximate to said outer surface.

3. The wrist sphygmomanometer as claimed in claim 1, further comprising a valve extending from said inflatable bladder and connected to said measuring unit, said measuring unit being operable to pump and release air into said inflatable bladder through said valve.
